(19) United States
(12) Patent Application Publication
(10) Pub. No.: US 2004/0028237 A1
(43) Pub. Date: Feb. 12, 2004

(54) Noise shaper for processing stereo signals

(75) Inventor: Haruhisa Yamaguchi, Kyoto-shi (JP)

Correspondence Address: SUGHRUE MION, PLLC, 2100 PENNSYLVANIA AVENUE, N.W., WASHINGTON, DC, 20037, US

(73) Assignee: ROHM CO., LTD.

(21) Appl. No.: 10/634,852

(22) Filed: Aug. 6, 2003

(30) Foreign Application Priority Data
Aug. 7, 2002 (JP) 2002-230417

(51) Int. Cl. A61F 11/06

(52) U.S. Cl. 381/71.14

(57) ABSTRACT

The invention intends to reduce the manufacturing cost of the noise shaper for processing stereo signals, to reduce the occupancy area of the circuit, and to reduce the power consumption of the noise shaper. In order to process a serial digital stereo signal in time-sharing, the noise shaper takes on a construction including: a conversion means that converts the inputted stereo signals into a serial time-division-multiplexed signal; an integration means that applies a delta sigma modulation to an inputted signal, in which integrators for integrating the inputted signal are connected in multi-stages; and a means that outputs to separate a noise shaped signal into right and left channel signals. Here, the integration means possesses an adding means, two storage means to which an output from the adding means is inputted, and a selection means that selects in time-sharing either of the outputs from the two storage means. And, the output of the selection means is fed back to the adding means.

### BACKGROUND OF THE INVENTION

1. Field of the Invention

The present invention relates to a noise shaper containing a delta sigma modulator used for processing stereo signals, specifically to a noise shaper having features in the delta sigma modulator.

2. Description of the Related Art

Conventionally, the processing of a digital stereo signal reproduced through a CD or DAT has been using the DA converter to which the over sampling technique and noise shaping technique are applied.

FIG. 5 illustrates a block diagram of a DA converter for stereo signals, to which a conventional over sampling technique and noise shaping technique are applied.

In the drawing, a two-channel digital stereo signal C is separated by an LR signal separation circuit 1 (hereunder, called I/F block). The separated signals each pass n-fold over sampling circuits 2, 2′ that separately eliminate aliasing noises and quantization noises, noise shapers 3, 3′ that output digital signals with a decreased number of bits to reduce noises in the lower frequency range, waveform shapers 4, 4′ that shape waveforms to remove noises, and LPFs 5, 5′ that filter the lower frequency components and convert the digital signals into analog signals; and through these circuits, the digital stereo signal C is converted into the right and left channel analog signals.

FIG. 6 illustrates a conventional circuit of the noise shaper. In the drawing, the noise shaper 3 is composed of an input area 31 that inputs the input signal into the noise shaper, and a delta sigma modulator 32, which converts inputted over sampled signals into delta sigma processed signals and output the result. The input area 31 is made up with a flip-flop 12; the delta sigma modulator 32 is made up with multipliers 15, e1, f1, and e2, flip-flops b1, b2, and adders a1, a2, and 16, and a comparator 18.

In this manner, the conventional DA converter for stereo signals is provided with the noise shapers each for the right channel and left channel separately, and the noise shaper needs a great many components. Accordingly, the manufacturing cost thereof is high, and the occupancy rate of space in the circuit is also high, which are the problems to be solved. Besides, the two noise shapers each use the power supply and the main clock independently, which leads to increasing the power consumption.

### SUMMARY OF THE INVENTION

This invention has been made in view of the above problems, and an object of the invention is to reduce the manufacturing cost of the noise shaper for processing stereo signals, to reduce the occupancy rate of area of the circuit, and to reduce the power consumption of the noise shaper. Another object of the invention is to reduce the power consumption during the processing of monophonic signals in replacement for stereo signals.

According to one aspect of the invention, the noise shaper for processing stereo signals includes: an input means that inputs two-channel stereo signals; a means that converts the two-channel stereo signals into a serial time-division-multiplexed signal; a delta sigma modulation means that inputs the serial time-division-multiplexed signal; and a means that outputs to separate a noise-shaped output signal into right and left channel signals.

Further, in the foregoing noise shaper for processing stereo signals, the delta sigma modulation means maybe provided with an integration means that is connected in a single stage or a multi-stage of two or more stages, which applies a delta sigma modulation to an inputted signal, in which the integration means is composed of: an adding means to which the serial signal is supplied, two storage means to which an output from the adding means is inputted in correspondence to the two channels, and a selection means that selects in time-sharing either of the outputs from the two storage means in correspondence to the two channels; and the output of the selection means is inputted to the adding means.

Further, in the noise shaper for processing stereo signals, the two storage means may be a flip-flop for the L-channel that operates on the basis of an L-channel clock, and a flip-flop for the R-channel that operates on the basis of an R-channel clock having a different phase with the L-channel clock.

According to the invention, one unit of the noise shaper for processing stereo signals applies the time-sharing to both the right and left channel signals; accordingly, in contrast to the conventional noise shaper composed of two units, the overlapped adders and multipliers and so forth can be eliminated from the circuit, the power consumption can be reduced to that extent, and the occupancy area of the circuit can be reduced, which contributes to further miniaturization of the circuit.

Especially when this noise shaper is applied to the DA converter, since it is generally known that as the order of the delta sigma modulator is higher, the characteristic thereof is better, the delta sigma modulator having a higher order can reduce the number of adders and multipliers to a greater extent.

Further, in case of implementing the monophonic processing, it can be realized by stopping either of the L-channel clock and R-channel clock. The power consumption of the clock in the monophonic mode can be lowered to about half the power consumption in the stereophonic mode.

### BRIEF DESCRIPTION OF THE DRAWINGS

FIG. 1 is a block diagram of a DA converter composed of a stereo signal processing noise shaper relating to the embodiment of this invention;

FIG. 2 is a waveform chart of signals in the noise shaper relating to the embodiment of this invention;

FIG. 3 is a circuit diagram of the noise shaper relating to the embodiment of this invention;

FIG. 4 is a block diagram of a control signal generator making up the DA converter in FIG. 1;

FIG. 5 is a block diagram of a conventional DA converter; and

FIG. 6 is a circuit diagram of a conventional noise shaper.

### DETAILED DESCRIPTION OF THE PREFERRED EMBODIMENTS

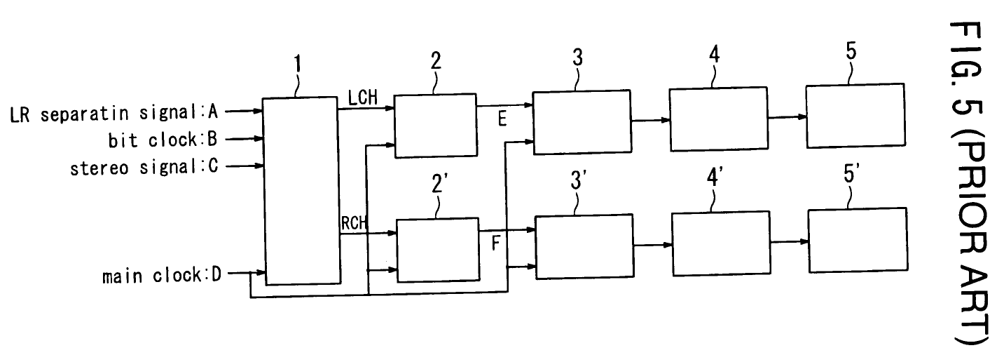

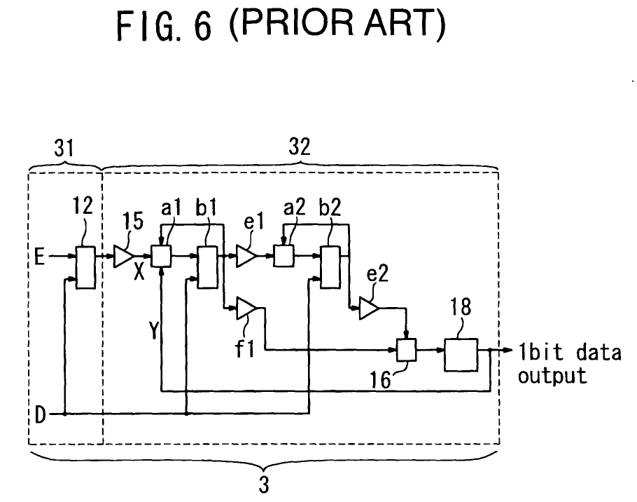

The preferred embodiments of the invention will be described with reference to the accompanying drawings. In the drawings, the same parts as those used in the descriptions of the conventional technique, illustrated in FIG. 5 and FIG. 6, are given the same symbols.

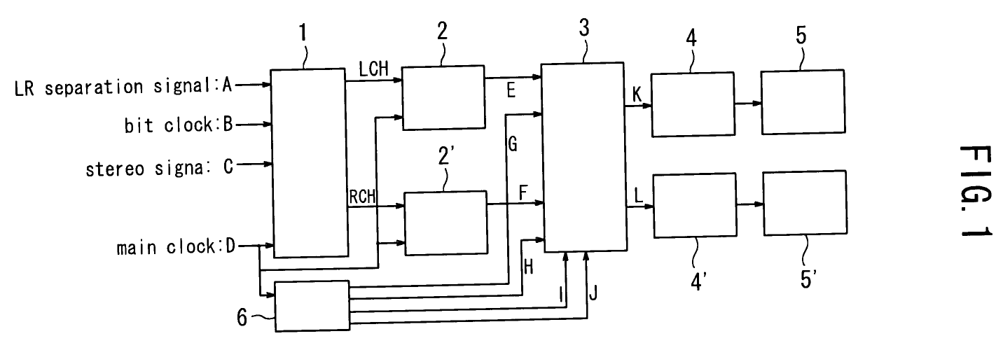

FIG. 1 illustrates the block diagram of a DA converter to which the noise shaper relating to the embodiment of the invention is applied.

In FIG. 1, the DA converter includes an LR signal separation circuit (hereunder, called I/F block) 1 to which a digital stereo signal C is supplied, n-fold over sampling circuits 2, 2′ to which output signals L, R separated by the I/F block are each supplied, a noise shapers 3 to which over sampled L/R channel signals are each supplied, waveform shapers 4, 4′ to which noised shaped L/R signals are each supplied, LPFs 5, 5′ to which wave-formed outputs from the waveform shapers are each supplied, and a control signal generator 6 that generates various control signals used in the circuits from the main clock through the noise shaper 3.

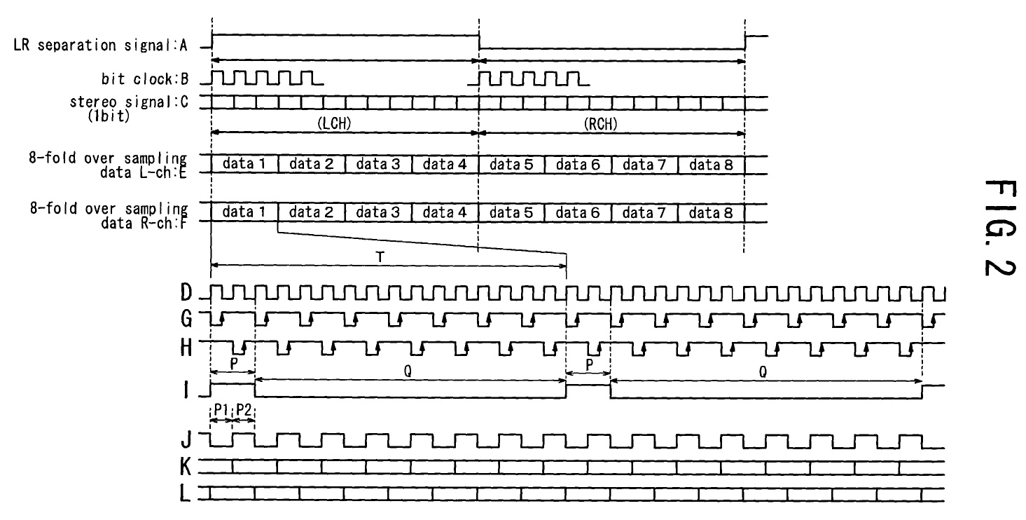

The operation of this DA converter will be described with reference to FIG. 2 illustrating the signal waveforms. The I/F block 1 captures the L-channel signal when the LR separation signal A is High, and captures the R-channel signal when it is Low, thereby separating the digital stereo signal C into right and left signals. Here, the bit clock B is to determine the timings of capturing the L-channel signal and R-channel signal each. Thus, the captured L/R channel signals are supplied to the over sampling circuits 2, 2′ in the following stage, which generate data signals E, F having the sampling frequency raised in order to reduce the aliasing noises and quantization noises of the captured L/R channel signals. Here in FIG. 2, the 8-fold over sampling circuit is shown as an example; however, this is not limited to 8-fold, it may be 16-fold, 64-fold, 128-fold, or even n-fold over sampling, which will be obvious to a person having ordinary skill in the art.

The noise shaper 3 executes the time division multiplexing to these data signals E, F, using the signals generated by the control signal generator, and thereby generates separation signals K, L of which lower band noises have been reduced by an LR time division signal J, L-channel clock G, and R-channel clock H, which are supplied to the waveform shapers 4, 4′ and LPFs 5, 5′.

The waveform shapers 4, 4′ execute the waveform shaping of the separation signals K, L to reduce noises, and then the LPFs 5, 5′ filter the lower components, whereby the separation signals K, L are converted into analog signals.

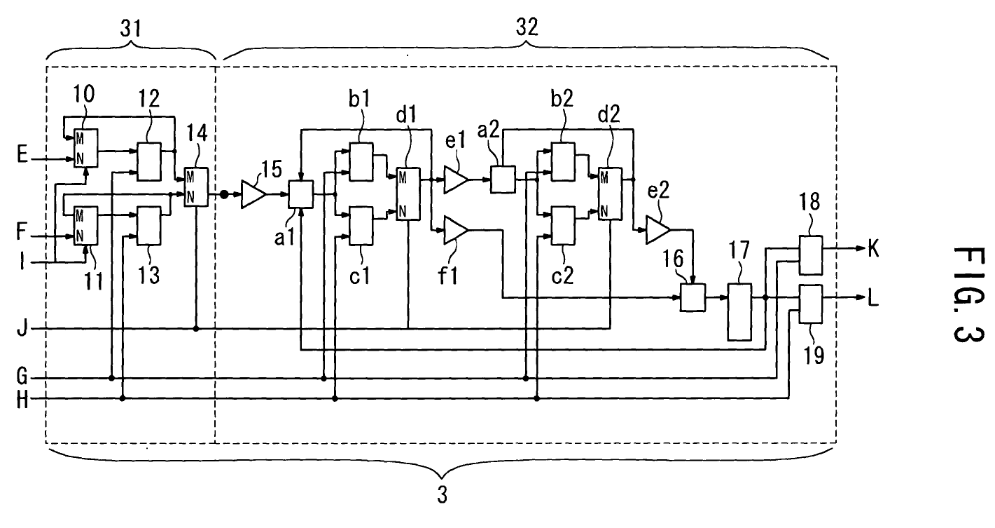

Referring to FIG. 3, the circuit diagram of the noise shaper 3 relating to the embodiment of this invention, the noise shaper 3 is composed of an input area 31 and a delta sigma modulator 32. The input area 31 is composed of selectors 10, 11 to which are inputted the L/R channel outputs E, F (data signals E, F) from the pre-stage over sampling circuit 2, flip-flops 12, 13 that hold the outputs from these selectors, and a selector 14 that selectively outputs the outputs of the flip-flops 12, 13.

The delta sigma modulator 32 is composed of a delta sigma modulation section that integrates an input signal, and an output section that outputs to separate a delta-sigma-modulated output signal into an L-channel signal and an R-channel signal.

The delta sigma modulation section is composed of first and second integrators that are cascaded. The first integrator includes a first adder al to which is supplied a signal having passed through the multiplier 15, an L-channel flip-flop b1 to which is supplied a signal from the first adder a1, which is operated by the L-channel clock G, an R-channel flip-flop c1 to which is supplied a signal from the first adder a1, which is operated by the R-channel clock H having a phase difference against the L-channel clock G, and a selector d1 that selects the outputs of the flip-flops b1, c1, in which the output from the selector d1 is fed back to the first adder a1.

The second integrator cascaded to the first integrator includes a second adder a2 to which is supplied an output signal from the first integrator, a second L-channel flip-flop b2 to which is supplied a signal from the second adder a2, which is operated by the L-channel clock G, a second R-channel flip-flop c2 to which is supplied a signal from the second adder a2, which is operated by the R-channel clock H having a phase difference against the L-channel clock G, and a second selector d2 that selects the outputs of the flip-flops b2, c2, in which the output from the selector d2 is fed back to the second adder a2.

The output signal from the second integrator passes through the multiplier e2, and the output signal from the multiplier e2 and the signal having the output from the first integrator multiplied by the multiplier f1 are added, and the added output is supplied to a comparator 17. The output from the comparator 17 is fed back to the first adder a1, which is also separated into an L-channel signal K and an R-channel signal L in the output section composed of flip-flops 18, 19.

Next, the operation of the noise shaper 3 thus configured will be described.

In the input area 31, the selectors 10, 11 capture the data signals E, F that are over-sampled at the pre-stage, by using a load signal I. The load signal I becomes High during a period P, and becomes Low during a period Q, of a data period T illustrated in FIG. 2. When the load signal I is High, the data are captured into the noise shaper 3; when it is Low, the input area 31 stops capturing data, and the noise shaper 3 executes the delta sigma modulation processing to the captured data. The data signals E, F are captured into the flip-flops 12, 13 by the L-channel clock G and the R-channel clock H that are generated by the control signal generator 4.

The selector 14 of the input area 31 outputs the L-channel signal and the R-channel signal captured by the load signal I as a serial digital stereo signal. That is, the data signals E, F are converted into a serial digital stereo signal being time-division-multiplexed by the input area 31.

Next, the operation of the delta sigma modulator 32 will be described. In each of the integrators constituting the modulator 32, while the LR time division signal J is Low, an L-channel processing terminal M is selected by each of the selectors d1, d2, and the L-channel data signal E is captured or integrated at the rise timing of the L-channel clock G. The results are held by the L-channel flip-flops b1, b2. That is, when the LR time division signal J is Low (P1), the delta sigma modulator 3 operates as a circuit that applies the delta sigma modulation to the L-channel data signal E. Similarly, when the LR time division signal J is High (P2), the delta sigma modulator 3 operates as a circuit that applies the delta sigma modulation to the R-channel data signal F.

The flip-flops 18 and 19 output to separate the above processed signal into the L/R separation signals K, L by means of the L/R channel clocks G, H.

In this manner, the noise shaper of this invention operates in time-sharing by means of the LR time division signal J, L-channel clock G, and R-channel clock H.

Here, the monophonic processing can be realized by stopping either of the L-channel clock and R-channel clock.

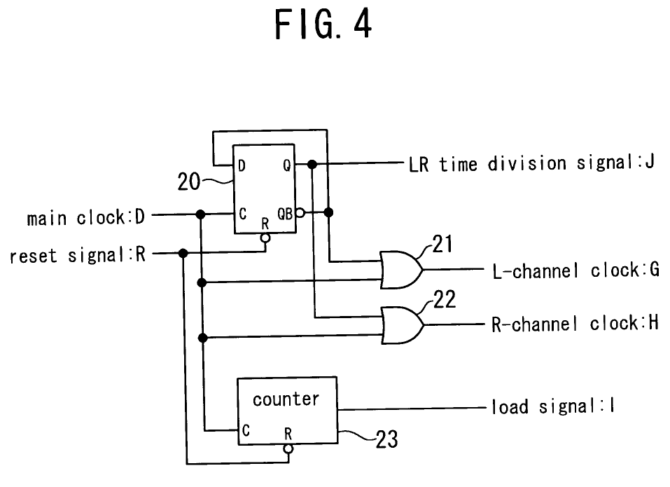

Referring to FIG. 4, the circuit configuration of the control signal generator 6 that generates the control signals will be described. The control signal generator 6 includes a D-type flip-flop 20 in which the C-terminal is supplied with the main clock, and the D-terminal receives a feedback from the QB-terminal, a counter 23, an OR circuit 22 that is supplied with the main clock D and the output from the Q terminal of the D-type flip-flop, and an OR circuit 21 that is supplied with the main clock D and the output from the QB terminal of the D-type flip-flop; thereby, the control signal generator delivers the LR time division signal J, L-channel clock G, R-channel clock H, and load signal I. Here, the reset signal R is served to reset the flip-flop 14 and the counter 23.

As it is clear from the comparison of the circuit diagram of the conventional DA converter illustrated in FIG. 5, which is already described, and the circuit configuration illustrated in FIG. 1, relating to the invention, while the conventional circuit contains two identical noise shapers, the circuit of the invention contains only one noise shaper, which is a novel feature of the invention.

Here in this embodiment, the order of the delta sigma modulator is assumed as the second, however it is not limited to the second order, but it may be any order. It is generally known that as the noise shaper takes a higher order, the characteristic thereof becomes better.

Further, the input area 31 of the conventional noise shaper is composed of one flip-flop (the noise shaper has two flip-flops since there are two input areas). On the other hand, the input area 31 of the noise shaper of the invention is composed of five circuit elements (two flip-flops and three selectors), which means that the circuit of the invention has more components in the input area 31. However, the number of overlapped components that can be eliminated in the delta sigma modulator 32 becomes larger in a higher order noise shaper; therefore, it is clear that the invention has a greater effect of reducing the number of components.

What is claimed is:

1. A noise shaper for processing stereo signals comprising:
an input means that inputs two-channel stereo signals;
a means that converts the two-channel stereo signals into a serial time-division-multiplexed signal;
a delta sigma modulation means that inputs the serial time-division-multiplexed signal; and
a means that outputs to separate a noise-shaped output signal into right and left channel signals.

2. A noise shaper for processing stereo signals as claimed in claim 1, wherein:
the delta sigma modulation means includes an integration means connected in a single stage or in a multi-stage of two or more stages, which applies a delta sigma modulation to an inputted signal;
the integration means includes: an adding means to which the serial signal is supplied, two storage means to which an output from the adding means is inputted in correspondence to the two channels, and a selection means that selects in time-sharing either of the outputs from the two storage means in correspondence to the two channels;
and the output of the selection means is inputted to the adding means.

3. A noise shaper for processing stereo signals as claimed in claim 2, wherein the two storage means are a flip-flop for the L-channel that operates on the basis of an L-channel clock, and a flip-flop for the R-channel that operates on the basis of an R-channel clock having a different phase with the L-channel clock.
